(19) United States
(12) Patent Application Publication
(10) Pub. No.: US 2003/0170333 A1
(43) Pub. Date: Sep. 11, 2003

(54) Cosmetic base complex

(76) Inventors: Karin Golz-Berner, Monaco (MC); Leonhard Zastrow, Monaco (MC); Olivier Doucet, Villefranche s/Mer (FR)

Correspondence Address: Stephan A. Pendorf, Pendorf & Cutliff, P O Box 20445, Tampa, FL, 33622-0445, US

(21) Appl. No.: 10/344,834

(22) PCT Filed: Aug. 14, 2001
(86) PCT No.: PCT/DE01/03071

(30) Foreign Application Priority Data
Aug. 15, 2000 (DE) 100 42 576.3

(51) Int. Cl. A61K 35/78; A61K 7/06; A61K 9/127

(52) U.S. Cl. 424/777; 424/74; 424/450

(57) ABSTRACT

The invention relates to a cosmetic base complex that contains extracts from fruit and daily products. Said base complex is characterized by an aqueous gel base which contains an encapsulated extract from an aqueous extraction of pineapple and the residue of an aqueous extraction of yogurt. The extractions are carried out in a temperature range of from 10 to 30° C. and the ratio of pineapple extract to yogurt residue ranges from 20:80 to 80:20. When used in a cosmetic preparation, the inventive complex may produce a certain feeling of happiness by increasing the serotonin and noradrenaline levels.

### BACKGROUND OF THE INVENTION

1. Field of the Invention

The invention relates to a cosmetic base complex that contains extracts from fruit and dairy products.

2. Description of the Related Art

A number of proposals have already been made for incorporating fruit or fruit extracts, including maritime plants and algae, into cosmetic products (DE-C-3515231 hafnia extract; DE-A-19824727 catechin and green tea).

It is also known to integrate milk or suitably prepared dairy products into cosmetic products. EP-A-839519, for example, discloses a cosmetic composition containing proteins and vitamins which contains 5-90% by weight of mammalian milk, 1-6% by weight of protein and yeast glucan.

### SUMMARY OF THE INVENTION

The object of the present invention is to develop a base complex which is made up of a combination of extracts from fruit and dairy products and which has a special effect exerted via the skin and via the complex's colour.

### DETAILED DESCRIPTION OF THE INVENTION

According to the invention, a cosmetic base complex is provided which consists of a gel base which contains an encapsulated extract from an aqueous extraction of pineapple fruit and the residue of an aqueous extraction of yogurt, the ratio of pineapple extract to yogurt residue ranging from 20:80 to 80:20 relative to their weight, and both extraction processes being carried out in a temperature range from 10 to 30° C.

The extract from pineapple fruit is obtained by means of an aqueous extraction at a temperature ranging from 10 to 30° C., preferably from 15 to 25° C. The extraction process is based on fresh fruit which is cut into pieces and brought into contact with water during a period of 5 to 24 hours while continuously being moved. Subsequently, the extract is filtered and encapsulated. The encapsulation can preferably be carried out using phospholipids so that usual liposomes result. In this way, the mixture of endopeptidases of the bromelain group, such as bromelin, ananase, inflamen, extranase, etc., obtained by means of the extraction and further enzymes are substantially included in the capsules in their entirety. In addition, the pineapple extract contains vitamins, e.g. vitamins C, B1, B2, B6, minerals such as high potassium contents combined with low sodium contents, chlorine, calcium, phosphorus, magnesium, iron, manganese, copper, zinc, iodine as well as serotonin.

As explained above, usual liposomes can be used as a transport system for the pineapple extract. Liposomes are completely closed membranes formed by lipid bilayers which enclose an aqueous volume. Liposomes can be unilamellar vesicles (i.e. have a single membrane bilayer) or multilamellar vesicles (i.e. onion-like structures characterized by several membrane bilayers each of which is separated from the next one by an aqueous layer). The bilayer consists of two lipid monolayers which have a hydrophobic “tail” area and a hydrophilic “head” area. The membrane bilayer is structured such that the hydrophobic (non-polar) “tails” of the lipid monolayer are oriented towards the centre of the bilayer, while the hydrophilic “heads” are oriented towards the aqueous phase.

The production of liposomes from saturated and unsaturated lipids has been described in a large number of patents, as has their use as a transport system. Advantageously, phospholipids are used as liposome forming agents in the present invention and production takes place in the usual way while at the same time the extract is included in the liposomes.

It is preferred that the starting material for the extraction of pineapple be fruit which has been cut into pieces. However, stems can also be processed as the active agents are contained in these parts of the plant as well.

Pure natural yogurt made from the milk of cows, sheep, goats or other animals or from mixtures of several kinds of milk is also subjected to an aqueous extraction process which takes place at a temperature ranging from 10 to 30° C., preferably from 15 to 25° C., and continues for a period of 5 to 24 hours. Subsequently, the aqueous phase is separated, e.g. by means of filtration, and an extraction residue obtained. The aforesaid extraction residue is then sterilized. The proteins, milk fats, sugars, folic acid, biotin, in some cases vitamins contained in the residue are preserved for further use. They will not be decomposed by the enzymes contained in the pineapple extract either as these are present in an encapsulated form.

The encapsulated pineapple extract and the yogurt residue are simultaneously mixed into an aqueous gel. In this way, a stable base complex is formed which serves as a starting material containing active agents for cosmetic formulations.

Suitable gel forming agents include Carbomer, Xanthan Gum, Carrageenan, Acacia Gum, Guar Gum, Agar-Agar, Alginate and Tylosen, Carboxymethyl Cellulose, Hydroxyethyl Cellulose, certain polyacrylates, Polyvinyl Alcohol, Polyvinylpyrrolidone, Montmorillonite. Carbomer, Guar Gum and Agar are preferred.

The complex according to the invention can be combined with usual cosmetic auxiliaries and carrier substances as they are generally used in such preparations, e.g. water, preservatives, vitamins, colourants, pigments which have a colouring effect, scavengers, thickeners, softening substances, moisturizing substances, fragrances, alcohols, polyols, esters, electrolytes, gel forming agents, polar and non-polar oils, polymers, copolymers, emulsifiers, waxes, stabilizers, glycerine. Usual O/W emulsions or W/O emulsions can also be produced using the aforesaid base complex, the oils which can be used including all usual oils such as mineral oils, synthetic oils, silicone oils as well as vegetable oils, such as Calendula Oil, Jojaba Oil, Avocado Oil, Macadamia Nut Oil, Castor Oil, Wheat Germ Oil, Grapeseed Oil, Kukui Nut Oil, Thistle Oil, Evening Primrose Oil, Safflower Oil, or a mixture of several thereof.

Further active substances can be added as well, e.g. kaolin according to WO96/17588 which has been modified with spherical TiO₂ or SiO₂ particles the particle size of which is <5 μm, the spherical particles making up 0.5 to 10% by weight of the kaolin mixture. In this way, the preparation makes the skin feel very soft and has an additional anti-inflammatory effect.

Further active agents are asymmetric lamellar aggregates which consist of phosholipids and fluorocarbon or a fluorocarbon mixture loaded with oxygen and whose fluorocarbon content is in the range from 0.2 to 100% by weight/volume while the phospholipid has a phosphatidylcholine content of more than 30 and up to 99% by weight according to WO94/00098.

Further cosmetic active agents include e.g. decomposition products obtained by subjecting yeast or vegetable matter to a gentle ultrasonic decomposition process according to WO94/13783; a product obtained by gentle decomposition of a vegetable or animal starting material in an aqueous medium and subsequent condensation with a substoichiometric amount of a C₁₀-C₂₀ fatty acid halogenide according to WO96/29048; a combination of active agents consisting of a) a product obtained by enzymatic extraction of the maritime plankton Artemia salina, which extraction product consists of phosphorylized nucleotides whose main component is diguanosin tetraphosphate; b) D-myo-inosit-1,4,5-triphosphate; c) glycan; the ratio of a:b:c being in the range of 1:0.1-50:0.1-30, according to WO99/38483; or a cosmetic preparation containing active agents which has a high radical protection factor according to WO99/66881.

The cosmetic product can also contain shellac in the aqueous phase, the content of pure shellac ranging from 0.1 to 20% by weight in an O/W emulsion, from 0.1 to 15% by weight in a W/O emulsion and from 0.1 to 10% by weight in a hydrogel. The aforesaid water soluble shellac is produced according to WO99/06010.

The cosmetic base complex according to the invention can e.g. be used in sun creams, sun gels, after sun products, day creams, night creams, masks, body lotions, cleansing milk, make ups, eye cosmetics, hair masks, hair conditioners, hair shampoos, shower gels, shower oils, bathing oils, lotions, gels. The aforesaid products are manufactured in a way which is known to those skilled in the art.

The ratio of the two complex forming agents is preferably in the range from 40:60 to 60:40.

The complex can be contained in a cosmetic formulation in an amount ranging from 0.5 to 10% by weight relative to the total weight of the formulation.

The base complex according to the invention contains valuable active agents which can develop their effect during a longer period of time once they have been applied on to the skin. The main special effect is a feeling of happiness which develops depending on the amount applied or the size of the body surface on to which the preparation is to be applied. The aforesaid feeling is not merely a feeling of happiness in the narrow sense, but a well balanced contentment, a feeling of relaxation coupled with pleasant sensations. The absorption of the product via the skin, in particular the supply of folic acid, seem to have an effect on the serotonin level and the production of noradrenaline though, there is no theory which proves this relationship.

In addition, a gentle peeling effect, i.e. an unnoticeable, even and gentle removal of upper particles of the skin, is achieved due to the content of the enzyme bromelin which by means of additives can be increased to a range of 0.1-1% by weight relative to the total weight of the cosmetic formulation.

Another aspect regarding the extract's and the base complex's effect is their bright yellow coloration brought about by the extraction of pineapple. It is known that such a bright yellow coloration also stimulates feelings of happiness so that a cosmetic formulation containing the base complex preferably has a bright yellow coloration.

The invention will hereinafter be explained in more detail by means of examples. All quantities are in % by weight if not indicated otherwise.

### EXAMPLE 1

### Emulsion

| Phase A | |
|---|---|
| Polyglyceryl-4 AlmondateSHEA | 3.5 |
| Butterate | |
| Paradecinol FV4.7 | 18 |
| Phase B | |
| Water | ad 100 |
| Glycerine | 3 |
| Magnesium sulphate | 0.5 |
| Phase C | |
| Base complex containing | |
| pineapple extract: | |
| yogurt residue in a ratio of 80:20 | 0.5 |
| Fragrance | 0.5 |
| Preservative | 0.8 |

Phase A and Phase B are produced separately by mixing the individual ingredients at a temperature of approx. 80° C. Subsequently, both phases are mixed with each other. Once the mixture has been cooled down to approximately 40° C., Phase C is stirred in.

### EXAMPLE 2

### Gel

| Phase A | |
|---|---|
| Water | ad 100 |
| Carbomer | 1.5 |
| Phase B | |
| Mineral Oil | 2 |
| Babassu Oil | 3 |
| EDTA | 0.1 |
| Phase C | |
| Triethanolamine | 1.5 |
| Phase D | |
| Base complex containing | |
| pineapple extract: | |
| yogurt residue in a ratio of 20:80 | 7 |
| Fragrance | 0.5 |
| Preservative | 1 |

Phase A is produced by mixing the individual ingredients and stirring thoroughly at a temperature of approx. 60° C., then Phase B is produced in the same way at a temperature of approx. 65° C. Then Phase C is added thus achieving a neutralization and finally Phase D is stirred in at a temperature of approximately 40° C.

The base complex is produced by mixing approximately equal parts by weight of water and fresh pineapple fruit which has been peeled and cut into pieces. The mixing process is continued during a period of 18 hours at a temperature of 20° C. Subsequently, the mixture is filtered and the filtrate is mixed with phospholipids at room temperature in order to achieve an encapsulation.

Further, 30 parts by weight of water and 70 parts by weight of a fresh natural yogurt made from cow's milk are mixed with each other by stirring thoroughly during a period of 20 hours at a temperature of 20° C. and the mixture is subsequently filtered. The filtrate is discharged and the residue is used for the production of the base complex.

Equal amounts of pineapple extract, which is present in the form of liposomes, and yogurt residue are simultaneously stirred into an aqueous gel (Carbomer) at a temperature of approx. 20° C. and the base complex obtained in this way is used as starting material for a cosmetic formulation. The base complex contains approx. 80% by weight of the extracts and approx. 20% by weight of the aqueous gel.

1. A cosmetic base complex which comprises
an aqueous gel base;
in the aqueous gel base, an encapsulated extract from an aqueous extraction of pineapple fruit at a temperature of 10-30° C.;
in the aqueous gel base, the residue obtained by an aqueous extraction of yogurt at a temperature of 10-30° C.; the ratio of pineapple extract to yogurt residue ranging from 20:80 to 80:20:

2. A base complex according to claim 1 wherein the pineapple extract is encapsulated in liposomes.

3. A base complex according to claim 2 wherein the liposomes are liposomes formed by phosholipids.

4. A base complex according to claim 1 wherein the ratio of pineapple extract to yogurt residue is in the range from 40:60 to 60:40.

5. A base complex according to claim 1 wherein the complex contains bromelin in the range from 0.1 to 1% by weight.

6. A base complex according to claim 1 wherein the said complex is contained in a cosmetic formulation in an amount ranging from 0.5 to 10% by weight relative to the total weight of the formulation.

7. A base complex according to claim 6 wherein the cosmetic formulation contains further components selected from the group consisting of oils, esters, moisturizing substances, solvents, pigments, emulsifiers, UV filters, antioxidants, vitamins, gel forming agents, further active agents and mixtures thereof.

8. A base complex according to claim 6 wherein the cosmetic formulation is present in the form of creams, gels, shower products, sun products, lotions, O/W emulsions, W/o emulsions or make ups.

9. A base complex according to claim 6 wherein the cosmetic formulation has a bright yellow colour.

10. Method for producing a cosmetic base complex which comprises the steps of producing an aqueous extract from pineapple at a temperature ranging from 10 to 30° C., subsequently filtering the said extract and encapsulating it in liposomes and mixing it into a cosmetic gel together with the residue of an aqueous extraction of yogurt carried out at a temperature ranging from 10 to 30° C.
